(19) United States
(12) Patent Application Publication
(10) Pub. No.: US 2004/0072213 A1
(43) Pub. Date: Apr. 15, 2004

(54) Compositions for DNA amplification, synthesis, and mutagenesis

(75) Inventors: Holly Hurlbut Hogrefe, San Diego, CA (US); Michael C. Borns, Escondido, CA (US); Michael L. Muhich, Olivenhain, CA (US)

Correspondence Address: FINNEGAN, HENDERSON, FARABOW, GARRETT & DUNNER, LLP, 1300 I STREET, NW, WASHINGTON, DC, 20005, US

(73) Assignee: Stratagene

(21) Appl. No.: 10/456,848

(22) Filed: Jun. 6, 2003

Related U.S. Application Data
(63) Continuation of application No. 09/414,295, filed on Oct. 6, 1999, now abandoned.

(51) Int. Cl. C12Q 1/68; C12P 19/34; C12N 9/22

(52) U.S. Cl. 435/6; 435/91.2; 435/199

(57) ABSTRACT

This invention provides compositions comprising a thermostable non-proofreading DNA polymerase, a thermostable proofreading DNA polymerase, and a factor that substantially inhibits the incorporation of undesired nucleotides or analogs thereof into a DNA polymer. The compositions may further comprise a buffer that enhances a polymerization reaction involving DNA polymerases. The invention also provides various methods of amplifying, synthesizing, or mutagenizing nucleic acids of interest using these novel compositions. Kits that comprise the compositions are also provided for amplifying, synthesizing, and mutagenizing nucleic acids.

The following patent applications and patent are hereby specifically incorporated by reference herein: U.S. patent application Ser. No. 08/957,709, filed Oct. 24, 1997; U.S. patent application Ser. No. 08/822,774, filed Mar. 21, 1997; International Application No. PCT/US98/05497, filed Mar. 20, 1998, published as International Publication No. WO 98/42860 on Oct. 1, 1998; U.S. Provisional Patent Application Serial No. 60/146,580, filed Jul. 30, 1999; U.S. patent application Ser. No. 08/164,290, filed Dec. 8, 1993; U.S. patent application Ser. No. 08/197,791, filed Feb. 16, 1994, which issued as U.S. Pat. No. 5,556,772 on Sep. 17, 1996; U.S. patent application Ser. No. 08/529,767, filed on Sep. 18, 1995.

### BACKGROUND AND SUMMARY OF THE INVENTION

The invention relates to the field of amplifying, synthesizing, and mutagenizing nucleic acids. Further, this invention relates to novel compositions and buffers for amplifying, synthesizing, or mutagenizing nucleic acids of interest.

In vitro polymerization techniques have enormously benefitted the fields of biotechnology and medicine. The ability to manipulate nucleic acids with polymerization reactions greatly facilitates techniques ranging from gene characterization and molecular cloning (including, but not limited to sequencing, mutagenesis, synthesis, and amplification of DNA), determining allelic variations, and detecting and screening of various diseases and conditions (e.g., hepatitis B).

An in vitro polymerization technique of great interest is the polymerase chain reaction (PCR). This method rapidly and exponentially replicates and amplifies nucleic acids of interest. PCR is performed by repeated cycles of denaturing a DNA template, usually by high temperatures, then annealing opposing primers to their complementary DNA strands, and then extending the annealed primers with a DNA polymerase. Multiple cycles of PCR result in an exponential amplification of the DNA template.

Unfortunately, PCR has limitations. These limitations range from 1) the rate of nucleotide incorporation, 2) the fidelity of nucleotide incorporation, 3) limitations on the length of the molecule to be amplified, and 4) the specificity of the polymerase.

Various methods to improve PCR exist. One approach is to optimize the reaction conditions, e.g., the reaction buffer, dNTP concentrations, or reaction temperatures. Another approach is to add various chemical compounds, e.g., formamide (Sarkar, G., et al. Nucl. Acids Res. 18: 7465, 1990), tetramethylammonium chloride (TMAC), or dimethyl sulfoxide (DMSO; Chevet et al., Nucl. Acids Res. 23:3343-3344, 1995; Hung et al., Nucl. Acids Res. 18:4953, 1990) to increase the specificity and/or the yield of the PCR reaction.

Other attempts include adding various proteins, such as replication accessory factors. Replication accessory factors known to be involved in DNA replication have also increased yields and the specificity of PCR products. For example, E. coli single-stranded DNA binding protein, such as SSB, has been used to increase the yield and specificity of primer extension and PCR reactions (U.S. Pat. Nos. 5,449,603, and 5,534,407). Another protein, the gene 32 protein of phage T4, appears to improve the ability to amplify larger DNA fragments (Schwartz et al., Nucl. Acids Res. 18:1079, 1990).

An important modification that has enhanced the ease and specificity of PCR is the use of Thermus aquaticus DNA polymerase (Taq) in place of the Klenow fragment of E. coliDNA pol I (Saiki et al., Science 230: 1350-1354, 1988). The use of Taq obviates any need for repeated enzyme additions, permits elevated annealing and primer extension temperatures, and enhances specificity. Further, this modification has enhanced the specificity of binding between the primer and its template. But, Taq has a drawback because it does not have 3′ to 5′ exonuclease (proofreading) activity and, therefore, cannot excise incorrect nucleotides added to the ends of the amplified products. Due to this limitation, the fidelity of Taq-PCR reactions typically have suffered. Therefore, those in the field have searched for alternative thermostable polymerases with proofreading activity.

Polymerases having proofreading activity have been found in archaea (archaebacteria). Archaea is a third kingdom, different from either the eukaryotes or the eubacteria. Many archaea are thermophilic bacteria-like organisms that can grow in extremely high temperatures, i.e., 100° C. One such archaea is Pyrococcus furiosus. A monomeric polymerase from Pyrococcus furiosus, referred to as Pfu polymerase or Pfu, has been identified that has the desired proofreading activity and synthesizes nucleic acids of interest at high temperatures (Lundberg et al., Gene 108: 1-6,1991; Cline et al., Nucl. Acids Res. 24: 3546-3551,1996). A second DNA polymerase has been identified in P. furiosus which has two subunits (DP1/DP2) and is referred to as P. furiosus pol II. (European Patent Application EP0870832, Kato et al., published Oct. 14, 1998; Uemori et al., Genes to Cells, 2:499-512, 1997). These polymerases may also be enhanced by accessory factors.

Certain natural proteins exist in archaea, i.e., PEF (polymerase enhancing factors) that exhibit deoxyuracil triphosphatase (dUTPase) activity and that enhance the activity of Pfu (published PCT Application No. WO 98/42860, Hogrefe et al., published on Oct. 1, 1998). The presence of deoxyuracil-containing DNA in a DNA polymerization reaction inhibits polymerase activity (Lasken et al., J. Biol. Chem. 271: 17692-17696, 1996). Specifically, during the course of a normal PCR reaction, a dCTP may be deaminated into dUTP, thereby introducing a deoxyuridine into the newly synthesized DNA (dU-DNA). When this newly synthesized DNA is thereafter amplified, the presence of the deoxyuridine inhibits Pfu. dUTP may also be present in some commercial dNTPs preparations. PEF substantially prevents dUTP incorporation and, thus, substantially avoids the inhibition of Pfu. Accordingly, PEF optimizes the activity of Pfu.

Another method for improving PCR is to use DNA polymerase blends which provide longer target-length capability, higher product yields, greater sensitivity, and faster cycling times than can be achieved with single enzyme formulations. Certain commercial blends presently available consist predominantly of Taq (or a related non-proofreading DNA polymerase), blended with a small amount of a proofreading enzyme such as Pfu. Two commercial DNA polymerase blends, marketed by Stratagene, are TaqPlus Long and TaqPlus Precision.

The TaqPlus Long PCR System, employing a mixture of Taq and Pfu and using 2 different buffers: low-salt (for 0-10 kb templates) and high-salt (for 10-18.5 kb templates), is suitable for amplifying genomic targets up to 18.5 kilobases (kb) and cloned targets up to 35 kb in length. The TaqPlus Precision PCR System was specifically formulated to provide high product yield and fidelity.

Another commercially available DNA polymerase blend is used in the Expand™ 20 kb^{plus} PCR System (Boehringer Mannheim). According to its manufacturer, it can amplify 20-40 kb genomic targets and lambda targets of greater than 40 kb. The Expand™ 20 kb^{plus} polymerase is a blend of Taq and Pwo (proofreading component) DNA polymerases.

Several mechanisms have been proposed to explain the role of the minor proofreading component in the commercial DNA polymerase blends for amplifying long DNA targets (“long PCR” blends). One explanation, proposed by Barnes, is that Pfu acts to correct mismatches that Taq cannot efficiently extend (Barnes, Proc. Natl. Acad. Sci. 91:2216-20, 1994). Such a proofreading role is supported by the observation that Pfu that substantially lacks 3′-5′ exonuclease activity (exo⁻ Pfu) typically cannot be used in place of Pfu in polymerase blends to amplify long targets.

Additionally, the use of two DNA polymerases may overcome limitations that arise due to one enzyme's inability to incorporate opposite abasic sites (or other lesions) or to carry out processive synthesis through regions of template secondary structure (Eckert and Kunkel, J. Biol. Chem. 268:13462, 1993). The DNA polymerase blends described by Barnes contained no more than 20% Pfu (U/U), and optimal performance was achieved with blends consisting of 0.1-1% Pfu (Barnes, Proc. Natl. Acad. Sci. 91:2216-20, 1994; U.S. Pat. No. 5,436,149). Blends containing a higher proportion of Pfu exhibited reduced product yield and target-length capability, which Barnes attributed to the presence of excess 3′-5′ exonuclease activity.

The efficiency of Pfu-catalyzed PCR reactions can be limited by the accumulation of dUTP during temperature cycling, and its subsequent incorporation into PCR products. Improvements in product yield and target-length capability of Pfu-catalyzed reactions can be achieved with the addition of a thermostable dUTPase, such as PEF, which prevents dUTP accumulation and incorporation into DNA (published PCT Application No. WO 98/42860, Hogrefe et al.). PCRs carried out with P. furiosus pol II are also enhanced by PEF.

Due to the problems encountered with certain presently available DNA polymerase blends (high non-proofreading component: low proofreading component), they typically are unable to efficiently amplify complex DNA templates longer than about 20 kb with high fidelity. There is a need for more advantageous thermostable DNA polymerase blends and novel compositions to provide high-fidelity amplification of longer DNA templates. There is also a need in the art for a universal reaction mixture capable of supporting synthesis, amplification, or mutagenisis of a broad range of DNA templates. The present invention addresses these needs.

According to certain embodiments, the invention provides compositions comprising: (a) a thermostable non-proofreading DNA polymerase, (b) a thermostable proofreading DNA polymerase, and (c) a factor that substantially inhibits the incorporation of undesired nucleotides or analogs thereof into a DNA polymer.

In certain embodiments, the invention provides compositions or buffers that enhance a polymerization reaction involving DNA polymerases.

In certain embodiments, the invention provides compositions wherein the amount of the proofreading DNA polymerase is greater than, or less than or about equal to, the amount of non-proofreading DNA polymerase, as determined by units of polymerase activity.

In certain embodiments, the factor that substantially inhibits the incorporation of undesired nucleotides or analogs is a dUTPase, for example a thermostable dUTPase. In certain embodiments, the thermostable dUTPase is PEF, SIRV dUTPase (Prangishvili et al., J. Biol. Chem. 273:6024-9, 1998), a thermostable archaeal dUTPase, a dUTPase from a thermophilic or hyperthermophilic eubacteria, or a dUTPase from a mesophilic organism.

In certain embodiments, the invention provides compositions further comprising one or more of the following additional components: PCR additives, including, but not limited to, betaine, glycerol, TMAC, polyethylene glycol (PEG), DMSO, gelatin, and/or non-ionic detergents; dNTP analogs, including, but not limited to, 7-deaza-2′-deoxyguanosine triphosphate, that destabilize DNA secondary structure; enzymes, including, but not limited to, enzymes such as pyrophosphatases or ligases; and replication accessory factors including, but not limited to, archaeal PCNA, archaeal RFC-P38, RFC-P55, archaeal RFA, and/or archaeal helicases, e.g., dna2 and helicases 2 to 8 (U.S. Provisional Patent Application No. 60/146,580). PCNA is a “sliding clamp” protein that stabilizes the interaction between the polymerase and the primed single-stranded DNA template and enhances synthesis of long DNA strands (also known as “processivity”) (Baker and Bell, Cell 92: 295-305, 1998). RFC is a “clamp-loading” protein complex that assembles the PCNA protein.

In certain embodiments, the compositions comprise an archaeal polymerase as the proofreading DNA polymerase. In certain embodiments, the non-proofreading DNA polymerase comprises Taq or a thermostable eubacterial polymerase.

In certain embodiments, the proofreading archaeal polymerase is Pfu or related family B or α-type polymerases found in other members of the archaea.

In certain embodiments, the proofreading archaeal polymerase is P. furiosus pol polymerase or related enzymes found in other members of the archaeal DNA polymerase II family.

In certain embodiments of the invention, the compositions comprise a proofreading archaeal DNA polymerase and a non-proofreading DNA polymerase, which may include either an eubacterial DNA polymerase or, alternatively, an archaeal DNA polymerase that substantially lacks 3′-5′ exonuclease activity, either naturally, or due to mutagenesis or modification.

In certain embodiments, the composition comprises Pfu as the proofreading DNA polymerase and Taq as the non-proofreading DNA polymerase. In certain embodiments, the Pfu:Taq ratio varies from about 1:1 to about 2 to 3:1, respectively, or even higher (i.e., greater than 3:1 Pfu:Taq). In certain embodiments the Pfu:Taq ratio is less than about 1:1, for example, but not limited to, Pfu:Taq ratios of about 1:1.01 to about 1:8.

In certain embodiments, the archael polymerase is related to Pfu or P. furiosus pol II. In certain embodiments, the archaeal polymerase is KOD (Toyobo), Pfx (Life Technologies Inc.), Vent (New England Biolabs), Deep Vent (New England Biolabs), Pwo (Roche Molecular Biochemicals), or JDF3 (Thermococcus sp. JDF3).

In certain embodiments, the invention provides methods for amplifying, synthesizing (including replicating), and mutagenizing nucleic acids of interest comprising using the novel compositions and buffers disclosed herein.

According to certain embodiments, kits are provided for amplifying, synthesizing, or mutagenizing nucleic acids of interest, comprising a non-proofreading DNA polymerase, a proofreading DNA polymerases, and a factor that substantially inhibits the incorporation of undesired nucleotides or analogs thereof into a DNA polymer. In certain embodiments, these kits further comprise a buffer that enhances a polymerization reaction involving the DNA polymerases and wherein the amount of proofreading DNA polymerase is greater than the amount of non-proofreading DNA polymerase, as determined by units of polymerase activity. In certain embodiments, kits are provided wherein the amount of the proofreading polymerase is less than or about equal to the amount of the non-proofreading polymerase, as determined by units of polymerase activity. It is understood by persons of skill in the art that the components of such kits may be provided either individually, i.e., in separate containers, or with at least two components combined prior to use in an amplification, synthesis, or mutagenizing reaction.

### BRIEF DESCRIPTION OF THE DRAWINGS

(All ratios are determined by units of polymerase activity.)

FIG. 1 illustrates PCR amplification of a 23 kb ββ-globin target in reaction buffer optimized for Pfu (50 mM Tricine, pH 9.1, 8 mM (NH₄)₂SO₄, 0.1% Tween 20, 2.3 mM MgCl₂ and 75 μg/ml nuclease-free bovine serum albumin (BSA)) using various Taq:Pfu DNA polymerase blend ratios, both with (lanes 1-4) and without PEF (lanes 5-8; 1 U/50 μl reaction). DNA polymerase blends used were 1.3:1 Taq:Pfu (lanes 1, 5), 1:1.25 Taq:Pfu (lanes 2,6), 1:1.8 Taq:Pfu (lanes 3,7), and 1:2.5 Taq:Pfu (lanes 4, 8). Lane M contains the LTI 5 kb marker.

FIG. 2 illustrates PCR amplifications comparing TaqPlus Long supplemented with 1 U/reaction PEF to a novel composition comprising a DNA polymerase blend of Pfu:Taq at a 2:1 ratio and PEF. All PCRs were carried out in a novel buffer (50 mM Tricine, pH 9.1, 8 mM (NH₄)₂SO₄, 0.1% Tween 20, 2.3 mM MgCl₂, and 75 μg/ml nuclease-free BSA). TaqPlus Long with PEF was used in lanes 1-3 and the novel composition was used in lanes 4-6. A human genomic DNA template was used at 240 ng (lanes 1, 4), 480 ng (lanes 2, 5), and 720 ng (lanes 3, 6). Lane M contains the LTI 5 kb marker.

FIG. 3 illustrates PCR amplification showing optimization of the ammonium sulfate (panel A) and DTT (panel B) concentrations in a preferred buffer (50 mM Tricine, pH 9.1, 0.1% Tween-20, 2.3 mM MgCl₂, and 75 μg/ml nuclease-free BSA). The following β-globin targets were amplified with 5U of the novel blend of 2:1 Pfu:Taq plus 1 U PEF, in the presence of various concentrations of ammonium sulfate: (A) 23 kb template (1) 8 mM, (2) 6 mM, (3) 4 mM, (4) 2 mM; 19 kb template (5) 8 mM, (6) 6 mM, (7) 4 mM, (8) 2 mM and (9) 2 mM. Lane M contains the LTI 5 kb marker. The following β-globin targets were amplified in the presence of various concentrations of DTT: (B) 23 kb template in 8 mM ammonium sulfate (1) 0 mM DTT, (2) 1 mM DTT, (3) 2 mM DTT, (4) 4 mM DTT, (5) 6 mM DTT; 23 kb template in 6 mM ammonium sulfate (6) 0 mM DTT, (7) 1 mM DTT, (8) 2 mM DTT, (9) 4 mM DTT, and (10) 6 mM DTT.

FIG. 4 illustrates PCR amplification showing a comparison of PCR amplification using TaqPlus Long with its optimal buffer (20 mM Tris HCI, pH 9.2, 60 mM KCl, and 2 mM MgCl₂) compared with PCR amplification using novel DNA polymerase blends and a novel optimized buffer (50 mM Tricine, pH 9.1, 8 mM (NH₄)₂SO₄, 0.1% Tween-20, 2.3 mM MgCl₂, 75 μg/ml nuclease-free BSA, and 2 mM DTT). Amplifications of the following β-globin targets were carried out: 17 kb (lane 1), 19 kb (lane 2), 23 kb (lanes 3, and 5-12), and 30 kb (lane 4). The polymerases and amounts used were: TaqPlus Long, 5U (lanes 1-4), a novel (optimal) blend of 2:1 Pfu:Taq, 5U (lane 5), 4U (lane 6), 3U (lane 7), 2U (lane 8); novel blend of 3:1 Pfu:Taq, 5U (lane 9), 4U (lane 10), 3U (lane 11), 2U (lane 12). All reactions carried out with a 2:1 or 3:1 Pfu:Taq polymerase blend included 1 U/reaction of PEF (lanes 5-12). Lane M contains Lambda/Hind III marker.

FIG. 5 illustrates PCR showing the effect of DMSO on amplifications of long genomic targets. In panel (A), amplifications of the 30 kb β-globin target were carried out in the presence of the following concentrations of DMSO: 0% (lane 1), 1% (lane 2), 2% (lane 3), or 3% (lane 4). In panel (B), the 26 kb β-globin target was amplified with the following concentrations of DMSO: 0% (lane 1) and 3% (lane 2). All PCR reactions were carried out using a 2:1 Pfu:Taq polymerase blend (5 U/reaction) and PEF (1 U/reaction) in a reaction buffer optimized for Pfu (50 mM Tricine, pH 9.1, 8 mM (NH₄)₂SO₄, 0.1% Tween-20, 2.3 mM MgCl₂, 75 μg/ml nuclease-free BSA, and 2 mM DTT). Lane M contains Lambda/Hind III marker.

FIG. 6 shows PCR amplifications of short genomic targets using the novel 2:1 Pfu:Taq blend with a novel buffer optimized for this blend (50 mM Tricine, pH 9.1, 8 mM (NH₄)₂SO₄, 0.1% Tween-20, 2.3 mM MgCl₂, 75 μg/ml nuclease-free BSA, and 2 mM DTT) or TaqPlus Long PCR with low salt buffer (20 mM Tris-HCl, pH 8.8, 10 mM KCl, 10 mM (NH₄)₂SO₄, 2 mM MgSO₄, 0.1% Triton X-100, and 100 μg/ml nuclease-free BSA). The following portions of human α1 anti-trypsin gene were amplified: 4 kb (panel A), 900 bp (panel B), and 105 bp (panel C). In panels A and B, PCRs were carried out with 2.5 U/reaction of TaqPlus Long (Taq:Pfu 16U:1U; lanes 1 and 2) or 2.5 U/reaction of the novel Pfu:Taq 2:1 blend with 1 U/reaction of PEF (lanes-3 or 4). In panel C, PCRs were carried out the novel Pfu:Taq 2:1 blend in the presence of 3% DMSO (lanes 1 and 2), 4% DMSO (lane 3), 5% DMSO (lane 4), and 0% DMSO (lanes 5-8). Lane M contains the Hinfl marker.

### DETAILED DESCRIPTION OF THE INVENTION

The following description should not be construed to limit the scope of this invention to any specifically described embodiment. Various aspects and embodiments of this invention will be apparent from the disclosure as a whole in context with the knowledge of one skilled in the art. In addition, the description herein, in combination with information known or available to persons of ordinary skill in the art, enables the practice of the subject matter encompassed by the following claims.

The invention provides novel compositions, comprising a thermostable non-proofreading DNA polymerase, a thermostable proofreading DNA polymerase, and a factor that substantially inhibits the incorporation of undesired nucleotides or analogs thereof into a DNA polymer, for amplifying a broad range of amplicon sizes, for example, but not limited to, genomic targets of 0.1 to 37 kb and up to 45 kb long for less complex lambda DNA targets. In certain embodiments, the compositions further comprise a buffer that enhances a polymerization reaction involving the DNA polymerases.

The term “substantially inhibits the incorporation of undesired nucleotides or analogs” means suppression of the incorporation of the undesired nucleotides or analogs to the point where the presence of incorporated undesired nucleotides or analogs does not materially affect the properties or yield of the polymerization reaction. For example, a dUTPase cleaves the dUTP molecule such that it is not incorporated into the DNA polymer. Proofreading DNA polymerases, in contrast, excise incorrect nucleotides or analogs that have already been incorporated into the DNA polymer. The term nucleotide “analog” refers to a molecule that is recognized by a polymerase and incorporated into the DNA polymer, excluding the dNTP that corresponds to the matching base on the template strand, for example A:T, G:C.

The terms “enhances a polymerization reaction” or “enhances a polymerization reaction yield” are intended to mean increasing the fidelity, increasing specificity, increasing synthesis through regions of template secondary structure, increasing the synthesis of long products, increasing the processivity, increasing the product yield and/or rate of nucleotide incorporation into a DNA polymer, compared to a polymerization reaction involving the DNA polymerases in a reaction buffer consisting of 20 mM Tris HCI, pH 9.2, 60 mM KCl, and 2 mM MgCl₂ The polymerization reaction may be enhanced, for example, by increasing the activity of the proofreading DNA polymerase, the non-proofreading DNA polymerase, or the factor that substantially inhibits incorporation of undesired nucleotides or analogs, either individually or in combination. The polymerization reaction may also be enhanced, for example, by increasing the activity of a replication accessory factor or other component of the inventive compositions or methods.

The use of “hot start” technology is within the scope of the claims. Hot start technology includes, but is not limited to, the use of reversibly-inactivated polymerases. Reversibly-inactivated DNA polymerases have been generated by either chemical or immunological inactivation. Immunological inactivation may result, for example, from combining neutralizing antibodies with a DNA polymerase. An example of a reversible chemical inactivation of DNA polymerases is described in Example 6 below.

Biological activity of the reversibly-inactivated polymerases can be recovered, e.g., by heating the chemically or immunologically inactivated component. Heating at appropriate temperatures can cause either the inactivating antibody molecules to be released or the chemical modification to be removed, resulting in the return of biological activity (Kellogg et al, BioTechniques 16:1134-37, 1994; Nieto et al., Biochim. Biophys. Acta 749:204-10, 1983). Commercial formulations of Taq with hot start capability, e.g., AmpliTaq Gold (Perkin Elmer) and Platinum Taq (Life Technology) are available. Other methods to reactivate reversibly-inactivated proteins include pH shift or other triggerable release mechanisms. It is expected that this reversible-inactivation technology will be applicable to numerous proteins, including, but not limited to, dUTPases, helicases, accessory factor proteins, various enzymes, etc.

According to certain embodiments, the invention provides compositions further comprising a thermostable proofreading DNA polymerase in a greater amount than the amount of thermostable non-proofreading DNA polymerase, as determined by units of polymerase activity. Also provided are compositions wherein the amount of the thermostable proofreading DNA polymerase is less than or about equal to the amount of the thermostable non-proofreading DNA polymerase, as determined by units of polymerase activity.

Certain embodiments of the invention provide compositions wherein the amount of thermostable proofreading DNA polymerase is greater than the amount of thermostable non-proofreading DNA polymerase, as determined by units of polymerase activity, and further comprising a buffer that enhances the polymerization reaction.

The terms “amplifying” or “amplification” refers to both linear and exponential amplification. Linear amplification includes, but is not limited to, non-cyclic primer extension. Examples of exponential amplification include, but are not limited to, polymerase chain reaction, rolling circle amplification, and strand displacement amplification.

The term “polymerase” as used herein is intended to encompass naturally-occurring or native polymerase, recombinantly-derived polymerase, as well as truncations, deletions, deriviatives, and variations of native or recombinantly-derived polymerase.

The term “units of polymerase activity” means the activity required to incorporate dNTPs into a template at the optimal temperature for that polymerase. One unit of polymerase activity is defined as the amount of polymerase needed to incorporate 10 nmoles of dNTPs in 30 minutes at the optimal temperature for that polymerase. Determining the optimal temperature for polymerase activity may require running a series of experiments in which all of the experimental conditions are held constant except reaction temperature, which is varied through a suitable range of temperatures. The optimal temperature would be that temperature at which maximum polymerization activity occurs. The optimal temperature for Pfu and Taq is 72° C.

For the purposes of determining the unit concentration of a polymerase used in PCR, polymerase samples are titrated in a series of PCR reactions. Unit concentrations are assigned based upon PCR performance relative to previously qualified lots of the same PCR enzyme (e.g., Pfu, Taq) (See Example 5).

When used in referring to dUTPases in general, or to PEF specifically, “units” means dUTPase units. One dUTPase unit is defined as the amount of enzyme which produces 1.757 nmole of inorganic pyrophosphate (“PPi”) per hour. The dUTPase assay (dUTP converted to dUMP+PPi) is carried out using the Sigma pyrophosphate reagent #P7275. The dUTPase, such as PEF, is incubated with 10 mM solutions of dUTP in 1× cloned Pfu buffer (20 mM Tris, pH 8.8, 10 mM KCl, 10 mM (NH₄)₂SO₄, 2 mM MgSO₄, 0.1% Triton X-100, 100 μg/ml nuclease-free BSA) for 1 hour at 85° C. PPi production is quantified as described in Sigma's Technical bulletin (Sigma Technical Bulletin No. BI-100, February 1999).

The term “hyperthermophilic” refers to organisms that grow optimally at temperatures of approximately 80° C.-85° C. and higher. The term “thermophilic” refers to organisms that grow optimally at temperatures of approximately 55° C.-85° C. The term “mesophilic” refers to organisms that grow at temperatures between approximately 15° C. and 45° C.

The term “thermostable,” when referring to a polymerase or a dUTPase, means an enzyme which is stable and active at temperatures from about 50-99° C. In certain embodiments, enzymes are stable at temperatures up to at least 90-99° C., such that the enzyme retains activity during temperature cycling, including at denaturation temperatures. In certain embodiments, thermostable enzymes will retain biological activity at temperatures between 68 and 95° C., the respective primer extension and template denaturation temperatures commonly used.

In certain embodiments, the novel compositions comprise an archaeal polymerase as the proofreading DNA polymerase. Thermostable archaeal DNA polymerase may be obtained from archaea such as Pyrococcus furiosus (Stratagene), Pyrococcus species GB-D (Deep Vent, New England Biolabs), Pyrococcus species strain KOD1 (Tagaki et al., AppI. Environ. Microbiol. 63:4504-10, 1997) (KOD, Toyobo; Pfx, Life Technologies), Pyrococcus woeseii (Pwo, Roche Molecular), Pyrococcus horikoshii, Pyrodictium occultum, Archaeoglobus fulgidus, Sulfolobus solfataricus, Sulfolobus acidocaldarius, Thermococcus litoralis (Vent, New England Biolabs), Thermococcus sp. 9 degrees North-7 (Southworth et al., Proc. Natl. Acad. Sci. 93:5281-5,1996), Thermococcus gorgonarius, Methanobacterium thermoautotrophicum, Methanococcusjannaschii, and Thermoplasma acidophilum, available from the American Type Culture Collection or the DSMZ German collection of Microorganisms and Cell Cultures. Related archaea from which the thermostable archaeal DNA polymerase may be obtained have been described (Archaea: A Laboratory Manual, Robb, F. T and Place, A. R., eds, Cold Spring Harbor Laboratory Press, 1995).

In certain embodiments, the non-proofreading DNA polymerase comprises a thermostable eubacterial polymerase. Thermostable eubacterial polymerases have been described in Thermus species (aquaticus, flavus, thermohilus HB-8, ruber, brokianus, caldophius GK14, filiformis), and Bacillus species (stearothermophilus, caldotenex YT-G, caldovelox YT-F). Commercial enzymes that are related to eubacterial pol I enzymes include Taq (Stratagene) Tth (Perkin Elmer), Hot Tub/Tfl (Amersham), KlenTaq (ClonTech), Stoffel fragment (Perkin Elmer), DynaZyme (Finnzymes), Bst (New England Biolabs), and Bca (Panvera). Thermostable pol III DNA polymerases have been described in Thermus aquaticus (Huang, et al. J. Mol. Evol. 48:756-69, 1999) and Thermus thermohilus (McHenry et al., J. Mol. Biol. 272:178-89, 1997), but could be obtained from other thermophilic eubacteria. Additional thermophilic eubacteria have been described (Kristjansson, J. K., Thermophilic Bacteria, CRC Press, Inc., 1992). Certain eubacterial pol I polymerases possess 3′-5′ exonuclease activity such as Thermotoga maritima pol I. Non-proofreading versions of such eubacterial polymerases could be used as the non-proofreading component in this invention. 3′-5′ exonuclease-minus versions (exo⁻) can be prepared by methods well-known in the art (Derbyshire et al., Methods of Enzymology 262:3-13, 1995).

In certain embodiments, the proofreading archaeal polymerase is P. furiosus pol II polymerase or homologous enzymes found in other members of the archaea. Archaea which contain genes that exhibit DNA sequence homology to P. furiosus pol II subunits have been described (Makinjemi, M. et al., Trends in Biochem. Sci. 24:14-16, 1999; Ishino et al., J. Bacteriol., 180:2232-6, 1998).

In certain embodiments, the factor that substantially inhibits the incorporation of undesired nucleotides or analogs thereof into a DNA polymer is a dUTPase. In certain embodiments, the factor is a thermostable dUTPase. In certain embodiments, the factor is a thermostable archaeal dUTPase. In certain embodiments, the factor is the archaeal dUTPase PEF. PEF can enhance several archaeal DNA polymerases, including Pwo from Pyrococcus woeseii, Deep Vent from Pyrococcus sp. GB-D, KOD from Thermococcus sp. KOD, Vent from Thermococcus litoralis and JDF-3 from Thermococcus sp. JDF-3. In contrast with the archaeal polymerases, PEF does not enhance the activity of Taq or other DNA polymerases of eubacterial origin. A broad range of PEF concentrations enhance PCRs carried out with DNA polymerase blends. A thermostable dUTPase from any thermophilic or hyperthermophilic eubacteria or archaea, or SIRV dUTPase, may enhance DNA polymerase blends containing an archaeal DNA polymerase as the proofreading component.

Polymerization reaction yields are increased when PEF is included with DNA polymerase blends containing archaeal polymerases. The activity and fidelity of these polymerase blends can also be increased by several methods, including increasing the proportion of the archaeal proofreading component in the blend, enhancing the activity of the archaeal proofreading polymerase by the addition of replication accessory factors, PCR additives, and optimizing the reaction buffer to enhance the polymerization reaction.

Various reaction buffers have been developed for use in long PCR reactions (i.e., amplification of templates of 20 kb or longer) using DNA polymerase blends that contain predominantly either the Taq or Tth eubacterial polymerases. Tris- or tricine-based reaction buffers (20-50 mM) are used, which exhibit a higher pH, i.e., 8.7-9.2 at room temperature, than standard PCR reaction buffers, and generally have a room temperature pH of 8.3-8.4. Buffers with higher pH have been proposed to enhance long PCR reactions by facilitating template denaturation and reducing DNA damage (Barnes, Proc. Natl. Acad. Sci. 91:2216-20, 1994; Cheng et al., Proc. Natl. Acad. Sci. 91:5659, 1994).

In addition, certain PCR additives that facilitate standard PCR have also been shown to enhance long PCR reactions, including DMSO (1-5%; Cheng et al., Proc. Natl. Acad. Sci. 91:5659, 1994), glycerol (5-8%; Cheng et al., Proc. Natl. Acad. Sci. 91:5659, 1994; Foord et al., in PCR Primer, eds. Dieffenbach and Dveskler, Cold Spring Harbor Laboratory Press, 1995), gelatin (0.01%; Foord et al., in PCR Primer, eds. Dieffenbach and Dveskler, Cold Spring Harbor Laboratory Press, 1995), and Tween 20 (0.05%; Foord et al., in PCR Primer, eds. Dieffenbach and Dveskler, Cold Spring Harbor Laboratory Press, 1995).

Potassium salts are commonly used in standard Taq- or Tth-based PCR reaction buffers at concentrations of 50 or 100 mM KCl, respectively. Cheng has reported, however, that lowering the potassium concentration (KCl, KOAc) by 1040% is more favorable for long PCR reactions using Taq- or Tth-based blends, although reduced specificity was noted (Cheng et al., Proc. Natl. Acad. Sci. 91:5659, 1994). Barnes has developed reaction buffers for KlenTaq-based polymerase blends (ClonTech), that used ammonium sulfate but not potassium (Barnes, Proc. Natl. Acad. Sci. 91:2216, 1994). As in standard PCR, Mg²⁺ is used for polymerase activity, and optimal concentrations used in long PCR reactions have ranged from 1.0-3.5 mM MgCl₂ or MgSO₄ (Barnes, Proc. Natl. Acad. Sci. 91:2216, 1994; Cheng et al., Proc. Natl. Acad. Sci. 91:5659, 1994; Foord et al., in PCR Primer, eds. Dieffenbach and Dveskler, Cold Spring Harbor Laboratory Press, 1995).

The reaction buffers developed for Taq- or Tth-based polymerase blends were found to be sub-optimal for enhancing blends containing Pfu and PEF. KCl was found to be inhibitory to Pfu and the addition of PEF did not overcome this inhibition. PEF enhancement was obtained when potassium salts were omitted from the reaction buffers.

According to certain embodiments of the invention, for Pfu-containing blends, a buffer is provided that contains 50 mM tricine, pH 9.1, 8 mM ammonium sulfate, 0.1% Tween 20, 2.3 mM MgCl₂, 75 μg/ml nuclease-free BSA, and 2 mM dithiothreitol (DTT). Certain components can be substituted for other reagents and the concentrations of some components can be varied, without substantially reducing performance in synthesis, mutagenizing, or amplification reactions.

In certain embodiments, the invention provides buffers and compositions comprising: about 20-70 mM Tricine or about 10-70 mM Tris-HCI; 0 to about 16 mM ammonium sulfate; about 0.01-0.2% Tween-20 or Triton X-100; between about 1.5 mM and 3 mM MgCl₂, MgSO₄, or C₄H₆O₄Mg; 0 to about 100 μg/ml nuclease-free BSA; and 0 to about 4 mM DTT. The preferred pH is between about 8.0 to about 9.5, more preferably between 8.4 to 9.2, and most preferably the pH is 9.1.

The addition of DMSO was also found to improve amplification of extra long nucleic acid targets using Taq/Pfu polymerase blends containing PEF. Concentrations of about 3-7% DMSO were optimal, but the optimal concentration was found to vary from system-to-system and with nucleic acid target size. DMSO concentrations of up to about 10% are believed to be useful in improving amplification of extra long templates. Similar organic compounds, including, but not limited to dimethylformamide (DMF), betaine, glycerol, or TMAC, could also be used to improve amplification of long nucleic acid targets (Landre et al., in PCR Strategies, M. Innis et al., eds., Academic Press, 1995; Henke et al., Nucleic Acids Research 25: 3957-3958, 1997).

It is expected that PEF will enhance DNA polymerase blends containing any archaeal polymerase as the proofreading component. The archaeal DNA polymerases Pwo, KOD, Vent, Deep Vent, JDF-3, and P. furiosus pol II are stimulated by PEF. Each of these enzymes may exhibit optimal activity in PCR reaction buffers containing different components and/or component concentrations than those found in the optimal Pfu reaction buffer. Using the buffer optimization procedures described herein, the skilled artisan will appreciate that optimal buffers for various thermostable proofreading DNA polymerases can be readily determined. Factors such as, but not limited to, pH, potassium ion concentration, ammonium sulfate concentration, reducing agents, stabilizing agents (e.g., but not limited to, proline, trehalose), and other buffer components, such as, but not limited to MOPS, HEPES, PIPES, may be important in optimizing a buffer to enhance polymerization reaction yield.

Although the DNA polymerase blends analyzed contained Taq as the non-proofreading component, other thermostable DNA polymerases substantially lacking 3′-5′ exonuclease activity may be utilized in PEF-containing blends. For example, PEF and a thermostable proofreading archaeal polymerase could be combined with any of a number of thermostable eubacterial polymerases. In addition to eubacterial DNA polymerases, the non-proofreading component may be an archaeal DNA polymerase that has been modified or mutated to eliminate the proofreading function. It has been previously demonstrated that an exo⁻Pfulexo⁺ Pfu blend could perform in long PCR, although typically not as well as KlenTaq I/Pfu polymerase blends (Barnes, Proc. Natl. Acad. Sci. 91:2216, 1996). Methods for preparing archaeal DNA polymerase mutants, with reduced or abolished proofreading activity, are well known in the art (See, e.g., Perler et al., Adv. Prot. Chem. 48:377, 1996).

The present invention also contemplates the use of blends of blends of DNA polymerases. For example, a formulation of three or more DNA polymerases, including at least one proofreading DNA polymerase and at least one non-proofreading DNA polymerase. Also contemplated by the present invention is the use of one or more factors that substantially inhibits the incorporation of undesired nucleotides or analogs thereof into a DNA polymer. Thus, in certain embodiments, the composition comprises one or more proofreading DNA polymerase, one or more non-proofreading DNA polymerase, and/or one or more factor.

The inventive methods, buffers, and compositions disclosed herein are also expected to be useful for synthesizing, amplifying, and mutagenizing nucleic acids at ambient and physiological temperatures, e.g., between about 20° C. and about 40° C. These applications would involve the use of DNA polymerases from mesophilic organisms. For example, but not as a limitation, the compositions may comprise a mesophilic proofreading archaeal DNA polymerase, obtained from Methanococcus voltae, and a mesophilic non-proofreading eubacterial DNA polymerase, such as exo⁻ Klenow fragment (Stratagene). It is apparent that numerous mesophilic proofreading and non-proofreading DNA polymerases found in the archaea, the eubacteria, bacteriophage, eukaryotic viruses and/or the eukaryotes could be successfully employed.

Certain embodiments of the invention are described in the following examples. However, these examples are offered solely for the purpose of illustrating the invention, and do not limit the invention.

### EXAMPLES

Methods

1. PCR Reaction Enzymes.

PCRs were carried out with DNA polymerase blends by: 1) adding the appropriate amounts of Pfu (Stratagene) and Taq (Taq2000, Stratagene) separately to the PCR buffer or by 2) combining Pfu (2.5 U/μl) and Taq (5 U/μl) at the appropriate ratios, and then adding an aliquot of the blend to the PCR buffers. dUTPase (PEF) was added separately to PCR reactions or reaction mixes to give a final concentration of 1 U/50 μl.

2. PCR Reaction Conditions.

PCR reactions were performed in 200 μl thin-walled PCR tubes using the appropriate PCR buffer. All targets ≧17 kb were amplified using 500 μM each dNTPs, 0-6% DMSO, 240 ng genomic DNA or 15-60 ng lambda DNA, and 4 ng/μl each primer in a 50 μl reaction volume. Water, buffer, dNTPs, primers, DNA, DMSO, Pfu:Taq (5U/reaction), and PEF (1U/reaction) were combined and gently mixed. Reactions were then overlaid with approximately 20 μl of mineral oil to prevent sample evaporation during prolonged cycling times. All targets ≦6 kb were amplified using 200 μM each dNTP, 0-3% DMSO, 100 ng genomic DNA or 15-60 ng plasmid DNA, and 2 ng/μl of each primer in a 50 μl reaction volume. All components were added and mixed as above, although no mineral oil was used.

3. Genomic DNA.

Three sources of genomic DNA were used:

A) Promega (catalogue # G304A)

B) ClonTech (catalogue #6550-1)

C) DNA isolated from cultured HeLa cells using the RecoverEase DNA isolation kit (Stratagene).

Wild type lambda DNA from Stratagene was isolated from purified lambda phage by phenol extraction and dialyzed against 10 mM Tris-HCI (pH 8.0), 1 mm EDTA. All DNA stocks were diluted to working concentrations (100 ng/μl genomic and 5 ng/μl lambda DNA) with distilled water (except the ClonTech product which was provided at 100 ng/μl) and stored at 4° C. to eliminate shearing due to repeated freezing/thawing.

4. Cycling Conditions.

The thermal cyclers used were the PTC-200 DNA Engine (MJ Research), the Model 9600 thermal cycler (Perkin-Elmer Biosystems), and the RoboCycler Gradient 96 (Stratagene). PCR profiles for each machine are as follows:

Profile A. Model 9600 and PTC-200.

| | ° C. | Time | #cycles |
|---|---|---|---|
| Denature | 92° C. | 2 min. | 1 |
| Denature | 92° C. | 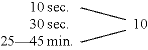 | |
| Anneal | 58-65° C. | | |
| Extension | 68-72° C. | | |
| Denature | 92° C. | 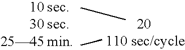 | |
| Anneal | 58-65° C. | | |
| Extension | 68-72° C. | | |

Profile B. Model 9600 and PTC-200.

| | ° C. | Time | # Cycles |
|---|---|---|---|
| Denature | 92° C. | 2 min. | 1 |
| Denature | 92° C. | 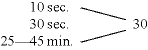 | |
| Anneal | 65° C. | | |
| Extension | 68° C. | | |
| Extension | 68° C. | 10 min | 1 |

Profile C. RoboCycler Gradient 96.

| | ° C. | Time | # Cycles |
|---|---|---|---|
| Denature | 92° C. | 2 min. | 1 |
| Denature | 92° C. | 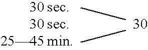 | |
| Anneal | 65° C. | | |
| Extension | 68° C. | | |
| Extension | 68° C. | 10 min. | 1 |

5. PCR Targets.

The following PCR targets were used to evaluate the performance of the amplification method using the novel 2:1 Pfu:Taq polymerase blend with PEF, in its novel buffer (50 mM Tricine, pH 9.1, 8 mM (NH₄)₂SO₄, 0.1% Tween-20, 2.3 mM MgCl₂, 75 μg/ml nuclease-free BSA, and 2 mM DTT):

Human β-Globin (Multi-Copy)

(SEQ ID NO: 1)

| | |
|---|---|
| NF19B | 5′-ATGCTATTAAAAACATGGACAACCTCAAGC-3′ |

(SEQ ID NO: 2)

| | |
|---|---|
| FβG | 5′-CACAAGGGCTAGTGGTTGGCGATT-3′ |

(SEQ ID NO: 3)

| | |
|---|---|
| F#3 | 5′-CTCAGATATGGCCAAAGATCTATACACACC-3′ |

(SEQ ID NO: 4)

| | |
|---|---|
| Rβg | 5′-AGGTTGCGAACGTGATCGCCTTTCTCCCAT-3′ |

(SEQ ID NO: 5)

| | |
|---|---|
| R54 | 5′-CAGGGGATTGACAGCAGTCTTCTCCTCAGG-3′ |

(SEQ ID NO: 6)

| | |
|---|---|
| NR56 | 5′-TATGGTTATGAGGAAACAGTCCAGGATCTC-3′ |

(SEQ ID NO: 7)

| | |
|---|---|
| R63 | 5′-ACAGCAAGAAAGCGAGGTTAGTGATACTTG-3′ |

Human tPA (Single Copy)

(SEQ ID NO: 8)

| | |
|---|---|
| F12-18 | 5′-GCTTCACTGTCTGCCTAACTCGTTCGTGTGTTCC-3′ |

(SEQ ID NO: 9)

| | |
|---|---|
| R18 | 5′-GCAGGGGTGCTGCAGAACTCTGAGCTGTACTTCC-3′ |

(SEQ ID NO: 10)

| | |
|---|---|
| R24 | 5′-TGTCTCCAGCACACAGCATGTTGTCGGTGAC-3′ |

F12-18 R18-18 kb

F12-18/R 24-24 kb

PCR Reaction Conditions for β-globin and tPA Targets

The 17-30 kb β-globin and 18 kb tPA were amplified together with the same profile using a 30-minute extension time (68° C.). For the 24 kb tPA fragment, a 40-minute extension time was used. For all targets, a 65° C. annealing temperature, 3% DMSO, 200 ng each primer (per 50 μl reaction), and 5U DNA polymerase blend (per 50 μl reaction) were used. Reactions were overlaid with approximately 20 μl mineral oil and cycled using amplification profiles A, B, or C (see above).

Lambda

| | |
|---|---|
| F211A | |
| 5′-CAGGTGGCTGAGATTTTGGGTG-3′ | (SEQ ID NO: 11) |
| R18505-22 | |
| 5′-CCGCGTTTACAATGTCCCCGAG-3′ | (SEQ ID NO: 12) |
| R25012-21 | |
| 5′-CCTGAATTTTCGGTGATGGCT-3′ | (SEQ ID NO: 13) |
| R30032-23 | |
| 5′-CGTGTTATCAAGCACTGCACTGG-3′ | (SEQ ID NO: 14) |
| R35130-22 | |
| 5′-GAATCAGCGCACATGGTACAGC-3′ | (SEQ ID NO: 15) |
| R40096-20 | |
| 5′-GCATCAGTAAGCGCATTGGC-3′ | (SEQ ID NO: 16) |
| R45047-21 | |
| 5′-GTTTGGGTTGTGCTGTTGCTG-3′ | (SEQ ID NO: 17) |

F211A/R18505-22-18 kb

F211 A/R25012-21-25 kb

F211 A/R30032-23-30 kb

F211A/R35130-22-35 kb

F211A/R40096-20-40 kb

F211A/R45047-21-45 kb

PCR Reaction Conditions for Lambda Targets.

The 18 kb and 25 kb lambda targets were amplified together with the same profile using a 25 minute extension time (at 68° C.). The 35 kb, 40 kb, and 45 kb lambda targets were amplified together with the same profile using a 40 minute extension time (at 68° C.). For all targets, a 58° C. annealing temperature, 200 ng each primer (per 50 μl reaction), and 5U DNA polymerase blend (per 50 μl reaction) were used. DMSO was added to final concentrations of: 3% (18 kb, 25 kb), 5-6% (35 kb, 40 kb), and 6% DMSO (45 kb). Reactions were overlaid with approximately 20 μl mineral oil and cycled using profiles A-C (see above).

Human α1 Anti-Trypsin

(SEQ ID NO: 18)

| | |
|---|---|
| F | 5′-CCTGAGGGCTCCCAGAGAGTGG-3′ |

(SEQ ID NO: 19)

| | |
|---|---|
| R | 5′-GGTTTAGCACGACCACAACAGC-3′ |

(SEQ ID NO: 20)

| | |
|---|---|
| F91-23 | 5′-GAGGAGAGCAGGAAAGGTGGAAC-3′ |

(SEQ ID NO: 21)

| | |
|---|---|
| R980-23 | 5′-GAGGTACAGGGTTGAGGCTAGTG-3′ |

(SEQ ID NO: 22)

| | |
|---|---|
| R2139-22 | 5′-GAAAATAGGAGCTCAGCTGCAG-3′ |

(SEQ ID NO: 23)

| | |
|---|---|
| R3979-22 | 5′-TTGGACAGGGATGAGGAATAAG-3′ |

(SEQ ID NO: 24)

| | |
|---|---|
| R6 | 5′ GAGCAATGGTGAAAGTCAAGGTCATCCACAGC-3′ |

F/R-105 bp

F91-23/R980-23-0.9 kb

F91-23/R2139-2.1 kb

F91-23/R3979-3.9 kb

F91-23/R6-6.0 kb

Lac I (pPRIAZ Plasmid)

(SEQ ID NO: 25)

| | |
|---|---|
| F155 | 5′-CATAGCGAATTGGCAAAACCTTTCGCGGTATGG-3′ |

(SEQ ID NO: 26)

| | |
|---|---|
| R156 | 5′-ACTACGGAATTCCACGGAAAATGCCGCTCATCC-3′ |

R155/F156-1.9 kb

PCR Reaction Conditions for α1 Anti-Trypsin and Lac I Targets.

All targets can be amplified without DMSO using 2.5 U of the indicated DNA polymerase blend and 100 ng of each primer per 50 μl reaction. The 0.9, 1.9, and 2.1 kb targets were amplified together with the same profile using a 2 minute extension time (at 72° C.), while the 3.9 kb and 6 kb targets were amplified together with the same profile using a 6 minute extension time (at 72° C.). For the 0.9-6 kb targets, a 58° C. annealing temperature was used. For the 105 bp target, the following cycling conditions were used for the RoboCycler gradient 96:

1 cycle at 94° C. for 1 min.

30 cycles at 94° C. for 40 sec.

57° C. for 40 sec.

72° C. for 1 min.

one cycle at 72° C. for 7 min.

### Example 1

Initial Optimization of Amplification Buffers for Taq:Pfu Polymerase Blends

Initial buffer optimization efforts focused on identifying the optimal buffer pH, the buffering component, and the ideal potassium salt composition. To identify the optimal parameters, PCR amplifications were performed in which one component was varied while the other components were held constant. When one component had been optimized, another component was varied, until the optimal levels for each component was determined.

The optimal pH of the tricine component was established by using three tricine stock solutions at pH 9.0, 9.1, and 9.3. Buffers made with tricine at pH 9.0 and 9.1 produced the highest yields, while reactions carried out at 9.3 produced lower yields.

In these same PCR reactions, buffers with Tween-20 were found to produce slightly higher yields than buffers with Triton X-100. An optimal concentration of 0.1% Tween-20 was identified.

The composition of the “prototype buffer” optimized for the TaqPlus Long polymerase blend supplemented with PEF included 50 mM Tricine (pH 9.1), 0-8 mM (NH₄)₂SO₄, 0.1% Tween-20, 2.3 mM MgCl₂, and 75 μg/ml BSA. PCR product yields were dramatically improved in the prototype buffer as compared to the TaqPlus Long high-salt buffer (20 mM Tris-HCl, pH 9.2, 60 mM KCl, 2 mM MgCl₂). Moreover, PEF-containing reactions produced dramatically higher yields than reactions lacking PEF when PCRs were carried out in the prototype buffer and the Expand™ 20 kb^{plus} buffer. Presumably lack of amplification in the TaqPlus Long high-salt buffer reflects KCl inhibition of the Pfu component of the TaqPlus Long polymerase blend, which could not be overcome with the addition of PEF.

### Example 2

### Optimization of Pfu:Taq DNA Polymerase Blends

Since PEF, and hence the Pfu component, had such a large influence on amplifications carried out in prototype buffer, it is possible that blends containing a higher proportion of Pfu would demonstrate even better performance than the TaqPlus Long DNA polymerase blend. Moreover, blends with a higher proportion of Pfu are expected to exhibit higher fidelity, as has been demonstrated for the TaqPlus Precision PCR System. With this in mind, novel DNA polymerase blends containing varying proportions of Pfu and Taq were tested (summarized in Table 1).

TABLE 1
DNA polymerase blend ratios of Taq to Pfu.

| Taq (U) | Pfu (U) | |
|---|---|---|
| 22 | 1 | |
| 18 | 1 | |
| 16 | 1 | |
| 14 | 1 | |
| 12 | 1 | |
| 10 | 1 | |
| 8 | 1 | |
| 3 | 1 | |
| 2 | 1 | |
| 1.4 | 1 | |
| 1.2 | 1 | |
| 1 | 1 | |
| 1 | 1 | |
| 1 | 1.25 | |
| 1 | 1.5 | |
| 1 | 1.6 | |
| 1 | 2 | (optimal Pfu:Taq ratio, 2:1) |
| 1 | 2.5 | |
| 1 | 3 | |
| 1 | 4 | |

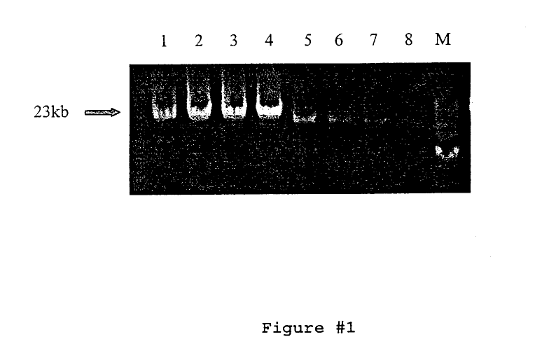

Amplifications of the 23 kb human β-globin target were carried out in the prototype buffer using a total of 5 U of each polymerase mixture (Taq U+Pfu U=5U) and 1 U of PEF per 50 μl reaction. Polymerase blend ratios of 12-22:1 (Taq:Pfu) generated similar moderate yields of amplicon, while ratios of 8-10:1 (Taq:Pfu) produced slightly higher yields. However, highest product yields were obtained using DNA polymerase blends with greater proportions of Pfu, ranging from 1-3:1 (Taq:Pfu) to 1:1.25-4 (Taq:Pfu). FIG. 1 shows that the prototype buffer supports successful amplification of the 23 kb β-globin target using blends that contain a higher proportion of Pfu compared to Taq (1:1.25-2.5 Taq:Pfu; lanes 24). Moreover, amplification was almost entirely dependent on the presence of PEF (FIG. 1; compare lanes 1-4 to 5-8). Blends including 1 U Taq to 2-3 U Pfu produced the highest yields overall (FIG. 1, lanes 3 and 4) and were therefore chosen for further analyses.

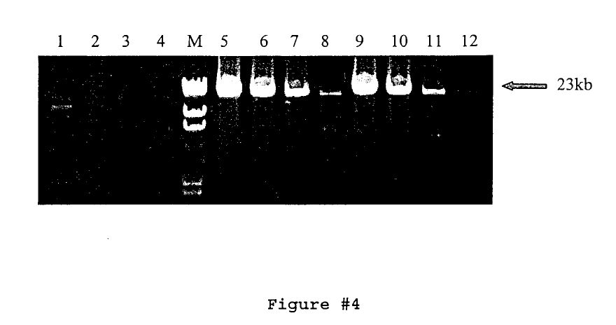

To determine the optimal Pfu:Taq polymerase blend composition, blends consisting of ratios 2:1, 2.5:1, and 3:1 (Pfu:Taq) were tested with a large panel of primer/template systems, including the 17, 19, 23, and 30 kb β-globin targets, and the 18 and 25 kb lambda targets. The blend ratio of 2:1 (Pfu:Taq) was found to synthesize the highest yields of the 23 and 30 kb β-globin targets, while all three blend ratios produced comparable yields for the 17 and 19 kb genomic targets and the 18 and 25 kb lambda targets. Based upon these results, the 2:1 Pfu:Taq ratio was determined to be optimal for DNA polymerase blends of Pfu and Taq. (FIG. 4).

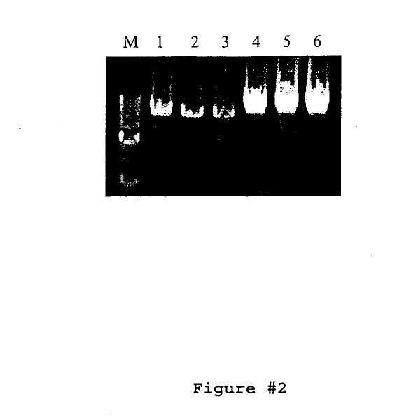

The 2:1 Pfu:Taq DNA polymerase blend produced significantly higher yields of the 23 kb β-globin target (FIG. 2, lanes 4-6) compared to the original TagPlus Long polymerase blend+PEF (FIG. 2, lanes 1-3) in the presence of PEF and the prototype buffer. Moreover, the 2:1 (Pfu:Taq) blend ratio appears to be less sensitive to high DNA template concentrations (FIG. 2, lanes 5 and 6), which can be inhibitory to TaqPlus Long+PEF reactions (FIG. 2, lanes 2 and 3).

### Example 3

### Buffer Optimization for 2:1 Pfu:Taq DNA Polymerase Blend

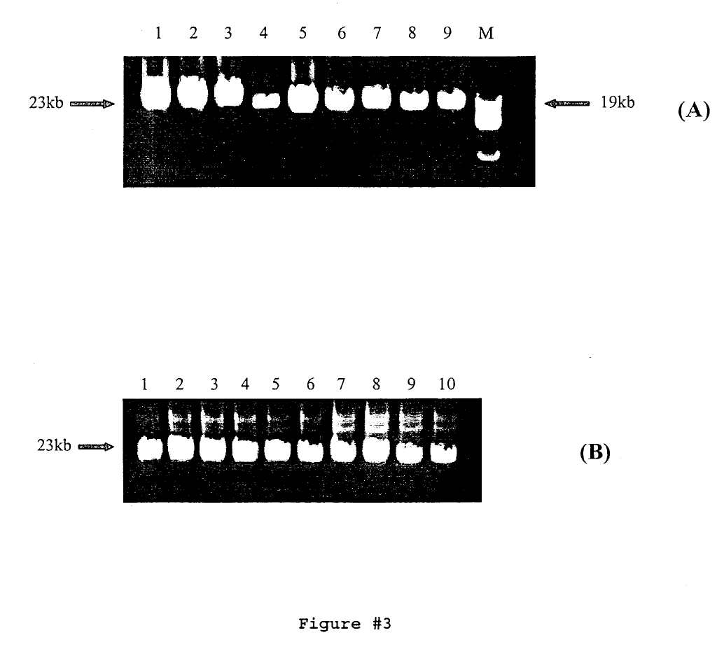

The PCR buffer composition was further optimized by optimizing the (NH₄)₂SO₄ and MgCl₂ concentrations and by evaluating the utility of reducing agents. It is presently unknown whether amplification of long targets is limited by oxidation of the DNA template and/or DNA polymerases. PEF was included in all of these reactions at 1 U/reaction. (NH₄)₂SO₄ concentrations of 0, 2, 4, 6, 8, 10 and 16 mM were tested in amplifications of the 19 kb and 23 kb β-globin targets. Polymerization reaction yields were highest in amplifications carried out in 4-8 mM (NH₄)₂SO₄ (FIG. 3A), while (NH₄)₂SO₄ concentrations >8 mM were inhibitory under the conditions tested.

In the particular reducing agent test performed, 2-mercaptoethanol was inhibitory, alone and in combination with DTT. By contrast, DTT enhanced product yields somewhat at concentrations of 1-2 mM (FIG. 3B). DTT titrations carried out in 8 mM (NH₄)₂SO₄ produced less background than titrations performed in the presence of 6 mM (NH₄)₂SO₄ (FIG. 3B, compare lanes 1-5 to 6-10). Therefore, concentrations of 8 mM (NH₄)₂SO₄ and 2 mM DTT were selected for the optimized buffer composition for use with the optimized Pfu:Taq DNA polymerase blend ratio of 2:1. MgCl₂ concentrations of 1, 1.5, 2, 2.3, 3, 3.5, 4, and 5 mM were tested in amplifications of the 17, 19, and 30 kb β-globin targets. MgCl₂ concentrations >2.3 mM were inhibitory under the conditions tested, and generated severe smearing, while amplifications carried out with <2 mM MgCl₂ concentrations were unsuccessful. All three targets were synthesized using MgCl₂ concentrations of 2 and 2.3 mM. A final concentration of 2.3 mM MgCl₂ was chosen because extra Mg²⁺ should support the addition of extra dNTPs or primers that may be required for synthesis of extremely long targets.

FIG. 4 illustrates the performance of the Pfu:Taq 2:1 DNA polymerase blend in the optimized Pfu:Taq buffer (50 mM Tricine, pH 9.1, 8 mM (NH₄)₂SO₄, 0.1% Tween-20, 2.3 mM MgCl₂, 75 μg/ml nuclease-free BSA, and 2 mM DTT). This novel composition, comprising the optimized Pfu:Taq 2:1 DNA polymerase blend in an optimized buffer containing PEF, produces dramatically higher yields of the 23 kb β-globin target (lane 5) compared to the TaqPlus Long PCR system (lane 3; in its high-salt buffer). Additional comparisons showed that while the TaqPlus Long PCR System can synthesize a 17 kb β-globin target in very low yield (lane 1), genomic targets of 19-30 kb were not amplified (lanes 2-4), consistent with the enzyme's target-length capability of 18.5 kb.

FIG. 4 also demonstrates that the optimal Pfu:Taq DNA polymerase blend ratio of 2:1 is superior to a 3:1 ratio when using the Pfu:Taq optimized buffer. Amplifications of the 23 kb β-globin target were performed using 2, 3, 4, or 5U (per 50 μl reaction) of the 2:1 or 3:1 ratios of the Pfu:Taq DNA polymerase blends. The optimal 2:1 Pfu:Taq DNA polymerase blend was found to produce higher polymerization reaction yields than the 3:1 blend (compare lanes 5-8 to lanes 9-12).

### Example 4

### Effect of DMSO on Target-Length Capability of the Optimized Pfu:Taq System

Using the 2:1 Pfu:Taq DNA polymerase blend with PEF in optimized Pfu:Taq buffer, the range of targets that could be amplified was tested. Initial efforts were directed to increasing amplicon length. The optimized Pfu:Taq buffer was shown to consistently produce high yields of the 17, 19, and 23 kb β-globin targets (see, e.g., FIGS. 3 and 4), and low yields of the 30 kb ββ-globin target. One of the potential limitations in amplifying long targets is the higher probability of encountering secondary structures in the DNA template that can block polymerase translocation. Structural impediments can form at lower annealing temperatures or can arise from incomplete melting of the template DNA, depending on local sequence context and % G-C content. PCR co-solvents and enhancers have been used to facilitate strand separation and melting of DNA templates (Landre et al., in PCR Strategies, Innis et al., eds., Academic Press, 1995; Sarker et al., Nucl. Acids Res. 18:7465, 1990; Henke et al., Nucl. Acids Res. 24:3957, 1997).

In an effort to enhance the target-length capability of the optimized Pfu:Taq buffer, several reagents were evaluated alone and in combination including DMSO, DMF, and betaine. In contrast to DMF and betaine, DMSO was found to consistently enhance product yields and amplification of long complex targets. Titration experiments were carried out with 14 different “long” PCR systems. The results, summarized in Table 2, indicate that optimal DMSO concentration varies from system-to-system, and increases with target length.

TABLE 2
DMSO concentration required for optimal PCR yield and
specificity.

| Target | % G/C | DMSO |
|---|---|---|
| 17 kb β-g | 37% | 3% |
| 18 kb tPA | 46% | 3% |
| 18 kb λ | 48% | 3% |
| 19 kb β-g | 37% | 3% |
| 23 kb β-g | 37% | 3% |
| 24 kb tPA | 46% | 3% |
| 25 kb λ | 48% | 5% |
| 26 kb β-g | 37% | 3% |
| 30 kb β-g | 37% | 3% |
| 30 kb λ | 48% | 5-6% |
| 35 kb λ | 48% | 5-6% |
| 37 kb β-g | 37% | 5% |
| 40 kb λ | 48% | 5-6% |
| 45 kb λ | 48% | 6-7% |

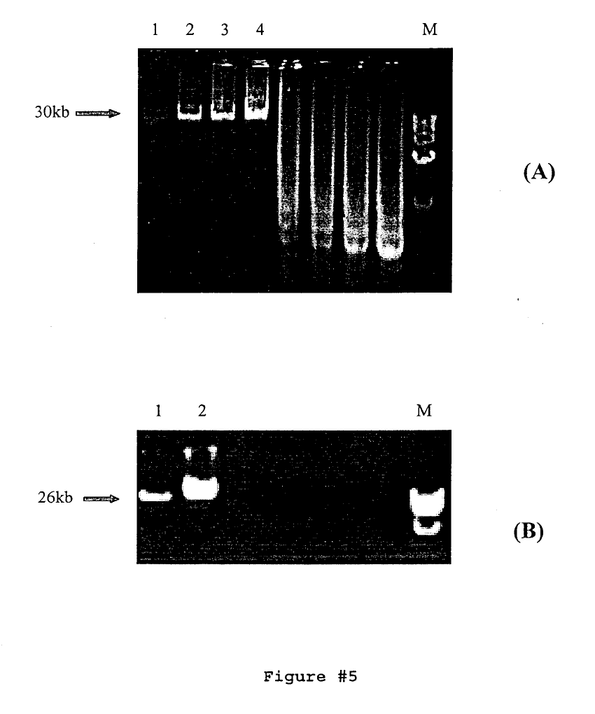

In total, the addition of DMSO resulted in high product yields with no background for lambda (“λ”) targets ≧30 kb, and provided the highest yields for β-globin (β-g) targets 17 kb to ≦30 kb in length (see, e.g., FIG. 5B). Addition of DMSO also provided synthesis of the tPA (18 kb, 24 kb) and ≧30-37 kb β-globin targets (FIG. 5A). For the 17-30 kb human β-globin targets, the most robust yields were generated with 3% DMSO, and concentrations >3% produced lower product yields (FIG. 5). Successful amplification of the 37 kb β-globin target was strictly dependent on the addition of 5% DMSO, and no product was generated with 4 or 6% DMSO. For the single-copy 18 kb and 24 kb tPA targets, 3% DMSO was also found to be optimal, and yields dropped off when higher concentrations were used. Variation in the DMSO concentration for optimal product yield and specificity presumably reflects a balance between template denaturation (favored by high DMSO) and efficient primer annealing (inhibited by DMSO). The higher DMSO requirement for the longest 37 kb β-globin target presumably reflects the increased difficulty of completely melting extra long DNA templates.

Essentially the same observations were made for the lower complexity lambda targets (48% G-C), except that even higher DMSO concentrations provided optimal specificity and yield. For the 25-45 kb lambda targets, highest yields of specific product were achieved with 5-7% DMSO, and the DMSO requirement increased with increasing target lengths. In long PCRs, the lambda DNA template appears to exhibit the tendency to produce shorter non-specific amplification products at DMSO concentrations of 0-4%. The use of 5-7% DMSO appears to relieve putative structural impediments, allowing the synthesis of specific full-length products.

As a single optimal DMSO concentration could not be identified for all target lengths, the optimal DMSO concentration should be determined for each target length. It is expected, for example, that DMSO titrations for genomic targets >17 kb will be between about 3-5% and for lambda targets >30 kb will be between about 5-7%.

### Example 5

### Polymerase Unit Determination Assays

The unit concentration of Pfu and Taq used to prepare the blends described herein can be determined by nucleotide incorporation assay or preferably by PCR titration assay.

Pfu PCR titration assay. PCR reactions (100 ul) consist of 1×cloned Pfu PCR buffer (20 mM Tris, pH 8.8, 10 mM KCl, 10 mM (NH₄)₂SO₄, 2 mM MgSO₄, 0.1% Triton X-100, 100 μg/ml nuclease-free BSA), 25 μM each dNTP, 75 ng each primer, 100 ng transgenic mouse genomic DNA, and cloned Pfu samples. The genomic DNA is from a transgenic mouse (LU or BK lineage; Kohler et al., Nucl. Acid Res. 18:3007-13, 1990) containing a lambda shuttle vector with an intact beta galactosidase gene.

The PCR primers have the following sequence:

| | |
|---|---|
| lambda (4266) | |
| 5′ GAG AGT CAC TCC GGC CCC 3′ | (SEQ ID NO: 27) |
| LacZ (19012) | |
| 5′ CGA CGA CTC GTG GAG CCC 3′ | (SEQ ID NO: 28) |

Amplifications are carried out with various amounts of the DNA polymerase to be assayed (e.g., new lot of cloned Pfu). At least 4 different dilutions are tested in amounts greater, equal to, and less than 2.5 U. The DNA polymerase to be assayed is diluted in Pfu final dialysis buffer (50 mM TrisHCl, pH 8.2, 0.1 mM EDTA, 1 mM DTT, 0.1% (v/v) Igepal CA 630, 0.1% (v/v) Tween 20, and 50% (v/v) glycerol). One μl of each polymerase dilution is added per 100 μl PCR reaction. In addition, positive control reactions are assembled which contain 2.5 U of previously qualified cloned Pfu DNA polymerase (Stratagene #600153). Reactions are carried out in duplicate.

The PCR reactions are cycled for 30 cycles using the following program:

94° C. 1 minute

54° C. 2 minutes

72° C. 90 seconds

The PCR reactions (35 μl) are electrophoresed on a 6% TBE gel. The gel is stained with ethidium bromide for 1 minute, and then destained for 1 minute (Molecular Cloning, Sambrook et al., Cold Spring Harbor Laboratory Press 1989). The gel is imaged using the Eagle Eye II Still Video System (Stratagene).

To determine the unit concentration of the DNA polymerase sample, one compares the yield of product amplified with 2.5 U of qualified cloned Pfu to the yields of product amplified with various amounts of the the test DNA polymerase. Unit concentration (U/μl) is determined by multiplying 2.5 U×the sample dilution which gives equal yield to 2.5 U cloned Pfu.

Additional PCRs (at least 3 systems) are performed to verify that 2.5 U of the test polymerase performs comparably to 2.5 U of Stratagene's cloned Pfu DNA polymerase.

Taq PCR titration assay. PCR reactions (100 μl) consist of 1×cloned Taq PCR buffer (10 mM Tris, pH 8.8, 50 mM KCl, 1.5 mM MgCl₂, 0.001% (v/v) gelatin), 25 μM each dNTP, 75 ng each primer, 100 ng mouse genomic DNA, and cloned Taq samples. The genomic DNA is from a transgenic mouse (LU or BK lineage; Kohler et al., Nucl. Acid Res. 18:3007-13, 1990) containing a lambda shuttle vector with an intact beta galactosidase gene.

The PCR primers have the following sequence:

| | |
|---|---|
| lambda (4266) | |
| 5′ GAC AGT CAC TCC GGC CCG 3′ | (SEQ ID NO: 29) |
| LacZ (19012) | |
| 5′ CGA CGA CTC GTG GAG CCC 3′ | (SEQ ID NO: 30) |

Amplifications are carried out with various amounts of the DNA polymerase to be assayed (e.g., new lot of cloned Taq). At least 4 different dilutions are tested in amounts greater, equal to, and less than 5 U. The DNA polymerase to be assayed is diluted in Taq final dialysis buffer (20 mM Tris-HCI, pH 8.0, 100 mM KCl, 0.1 mM EDTA, 1 mM DTT, 0.5% (v/v) Igepal CA 630 (or NP40), 0.5% (v/v) Tween 20, and 50% (v/v) glycerol). One μl of each polymerase dilution is added per 100 μl PCR reaction. In addition, positive control reactions contain 5 U of previously qualified cloned Taq (Taq2000 #600195, Stratagene). Reactions are carried out in duplicate.

The PCR reactions are cycled for 30 cycles using the following program:

94° C. 1 minute

54° C. 2 minutes

72° C. 1 minute 30 seconds

The PCR reactions (35 μl) are electrophoresed on a 6% TBE gel. The gel is stained with ethidium bromide for 1 minute, and then destained for 1 minute.

The gel is then imaged using the Eagle Eye II Still Video System.

To determine the unit concentration of the DNA polymerase sample, one compares the yield of product amplified with 5 U of qualified cloned Taq DNA polymerase to the yields of product amplified with various amounts of the test DNA polymerase. Unit concentration (U/μl) is determined by multiplying 5U×the sample dilution which gives equal yield to 5 U cloned Taq2000.

Additional PCRs (at least 3 systems) are performed to verify that 5 U of the unknown polymerase performs comparably to 5 U of Taq2000 DNA polymerase.

Incorporation assay. Polymerase unit concentrations can be determined by nucleotide incorporation assay. The person of skill in the art will recognize that many variations on the following assay will provide similar results.

DNA polymerase activity is measured using activated calf thymus DNA. A typical DNA polymerase reaction cocktail contains: 50 mM Tris-HCI, pH 8.0, 5 mM MgCl₂, 1 mM DTT, 50 μg/ml BSA, 4% glycerol, 200 μM each dATP, dCTP, dGTP, 195 μM TTP, 5 μM [³H]TTP (New England Nuclear #NET-221H, 20.5 Ci/mmole; partially evaporated to remove EtOH), 250 μg/ml of activated calf thymus DNA (e.g., Pharmacia #27-4575-01)

Polymerases are serially diluted in appropriate storage buffers and 1 μl of each enzyme dilution is added to 10 μl aliquots of polymerase cocktail. Polymerization reactions are conducted in duplicate or triplicate for 30 minutes at optimal temperature. The extension reactions are quenched on ice, and then 5 μl aliquots are spotted immediately onto DE81 ion-exchange filters (2.3 cm; Whatman #3658323). Unincorporated [³H]TTP is removed by 6 washes with 2×SCC (0.3 M NaCl, 30 mM sodium citrate, pH 7.0), followed by a brief wash with 100% ethanol. Incorporated radioactivity is measured by scintillation counting. Reactions that lack enzyme are also set up along with sample incubations to determine “total cpms” (counts per minute; omit filter wash steps) and “minimum cpms” (wash filters as above). The bound cpms is proportional to the amount of dNTPs incorporated, and can be converted into units of DNA polymerase activity. One unit of polymerase activity is defined as the amount of enzyme that catalyzes the incorporation of 10 nmoles of total dNTP into polymeric form (binds to DE-81 paper) in 30 minutes at the optimal temperature of 72° C. To determine units, background (average “minimum cpms” value) is first subtracted from the average sample cpms. Units of polymerase activity can then be calculated using the following equation:

$$\frac{( corrected sample cpms )}{total cpms} \times \frac{( 8 nmoles dNTPs )}{reaction} \times \\ \frac{( 1 unit )}{( 10 nmoles dNTPs incorporated )} = Units of Poly \text{-} merase Activity$$

Polymerase concentrations (U/ml) can be extrapolated from the slope of the linear portion of units versus enzyme volume plots (linear range is generally 0.03-0.003 U).

### Example 6

### Reversible Chemical Inactivation of DNA Polymerases

Reversibly inactivated versions of DNA polymerases (Taq, Pfu) have been prepared using citraconic anhydride. Polymerase samples are incubated at room temperature for at least one hour with a 150-500 fold molar excess of citraconic anhydride. The citraconic anhydride is first diluted in dioxane and then added to polymerase samples in 50 mM potassium phosphate buffer (pH 7.9; final dioxane concentration is <4%). Inactivated samples are then dialyzed against several changes of suitable enzyme storage buffers, such as 20 mM Tris-HCI, pH 8.0, 100 mM KCl, 10 mM DTT, 50% glycerol, 0.5% Igepal CA630, and 0.5% Tween 20 (for Taq). Complete inactivation is confirmed by the lack of detectable nucleotide incorporation into activated DNA after approximately 1 hour at 72° C. (sample cpms=minimum cpms). See example 5 “incorporation assay” for a description of a suitable nucleotide incorporation assay. To assess reversibility of citraconic anhydride modification, inactivated polymerase preparations are heated at 95° C. for at least 10 minutes, and then tested for nucleotide incorporation activity. The efficiency of regeneration is influenced by buffer components, particularly pH, where regeneration is enhanced as the pH is lowered (e.g. pH 8.3 is better than 8.8). Therefore, buffer optimization typically can accomplish both efficient regeneration of active polymerases (and/or dUTPase) and optimal PCR using novel polymerase blend plus dUTPase compositions.

Other Important Parameters for Developing Optimal Amplification Buffers using DNA Polymerase Blends.

Several additional parameters (other than DMSO) typically are important in achieving optimal long-target amplification. These parameters include: 1) quality of the DNA template, 2) dNTP concentration, 3) amount of DNA polymerase, 4) cycling parameters, and 5) DNA template concentration. These parameters are described in the following paragraphs.

1. Quality of DNA Template.

The purity and integrity of the DNA template typically has an important impact on amplification of extra long targets, for example, templates longer than 20 kb. As an illustration, amplifications of the 23 kb β-globin target were carried out using three different sources of human genomic DNA. Of the commercial preparations, genomic DNA manufactured by Promega was found to be superior to that of ClonTech. Genomic DNA prepared from HeLa cells using the RecoverEase kit (Stratagene) was also found to exhibit superior performance in long PCR reactions, producing somewhat higher yields than obtained with Promega's genomic DNA.

2. dNTP Concentration.

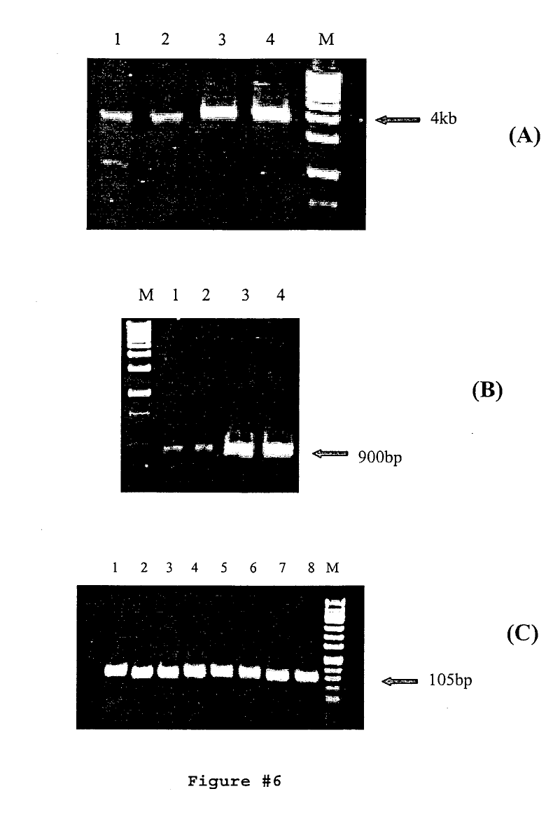

dNTP concentration was found to have an important effect on the efficiency of amplification. For targets ≧18 kb, 500 μM of each dNTP was optimal. Higher concentrations, up to 750 μM, had neither a positive nor negative effect. Lower amounts resulted in decreased amplicon yields. For targets ≦6 kb, 200 μM of each dNTPs was optimal (FIG. 6), and the use of higher concentrations (up to 500 μM tested) resulted in lower PCR product yields.

3. Amount of DNA Polymerase.

As indicated in FIG. 4, the amount of enzyme added per reaction had a significant effect on amplicon yields. For targets ≧18 kb, robust yields are typically produced using 5 U of the 2:1 Pfu:Taq DNA polymerase blend per 50 μl PCR reaction. This amount works well with complex, high-copy number targets up to 30 kb and for low-complexity targets up to 45 kb. Amounts up to 10 U/50 μl PCR reaction can generate even higher yields and work well with complex, low-copy number targets ≧24 kb and complex, high-copy number targets ≧30 kb in length. For all targets ≦6 kb, 2.5 U/50 μl PCR reaction was found to be optimal, and the use of 5 U/50 μl PCR reaction resulted in severe loss of amplicon yield.

4. Cycling Parameters.

Robust yields can be generated with a variety of PCR profiles and thermal cyclers, demonstrating the versatility of optimal DNA polymerase blends. For the most part, PCR profiles A-C could be used interchangeably for all of the PCR systems investigated. For the 30-37 kb genomic targets, somewhat higher yields could be produced with profile A, which includes the addition of 10 seconds per cycle in cycles 11-30, but higher yields were not consistently observed in the majority of experiments.

For all targets, an extension time of 1 minute per kb was sufficient to produce high product yields. Longer extension times generally produce higher yields, but are most important for low copy number, complex targets ≧24 kb. Extension temperature typically also has a significant effect on amplicon yield. For targets ≧18 kb, a 68° C. extension temperature was optimal, and when extension temperatures were raised to 72° C., a sharp decrease in amplicon yield was observed. The opposite was true for targets ≦6 kb in length, where optimal results were obtained using 72° C. extension temperatures.

5. DNA Template Concentration.

The amounts of genomic DNA optimal for amplifications with the optimized Pfu:Taq formulation are: 100-250 ng/50 μl reaction (for targets ≧6 kb) and 250 ng-1 μg (for targets ≧6 kb). Successful amplification of complex targets has been achieved using as much as 780 ng of genomic DNA per 50 μl reaction (FIG. 2A). For low-complexity targets, typically the effective concentrations are 15-60 ng of lambda or plasmid DNA.

All documents mentioned in this application, including but not limited to, articles, books, reviews, patents and patent applications, are hereby incorporated by reference in their entirety into this specification.

What is claimed is:

1. A composition comprising: (a) a thermostable non-proofreading DNA polymerase, (b) a thermostable proofreading DNA polymerase, and (c) a factor that substantially inhibits the incorporation of undesired nucleotides or analogs thereof into a DNA polymer.

2. The composition of claim 1, wherein the amount of the proofreading DNA polymerase is greater than the amount of non-proofreading DNA polymerase, as determined by units of polymerase activity.

3. The composition of claim 1, wherein the amount of the proofreading DNA polymerase is less than or about equal to the amount of non-proofreading DNA polymerase, as determined by units of polymerase activity.

4. The composition of claim 1, further comprising a buffer that enhances a polymerization reaction involving the DNA polymerases.

5. The composition of claim 1, wherein the amount of the thermostable proofreading DNA polymerase is greater than the amount of the thermostable non-proofreading DNA polymerase, as determined by units of polymerase activity, and further comprising a buffer that enhances a polymerization reaction involving the DNA polymerases.

6. The composition of claim 1, wherein the factor is a dUTPase.

7. The composition of claim 1, wherein the factor is a thermostable dUTPase.

8. The composition of claim 7, wherein the thermostable dUTPase is archaea dUTPase.

9. The composition of claim 8, wherein the archaeal dUTPase is PEF.

10. The composition of claim 7, wherein the thermostable dUTPase is from a thermophilic or hyperthermophilic eubacteria.

11. The composition of claim 1, wherein the thermostable proofreading DNA polymerase is an archaeal DNA polymerase.

12. The composition of claim 1, wherein the thermostable proofreading DNA polymerase is Pfu.

13. The composition of claim 1, wherein the thermostable non-proofreading DNA polymerase is an eubacterial DNA polymerase.

14. The composition of claim 1, wherein the thermostable non-proofreading DNA polymerase is Taq.

15. The composition of claim 1, wherein the thermostable non-proofreading DNA polymerase is an archaeal DNA polymerase that substantially lacks 3′-5′ exonuclease activity.

16. The composition of any of claims 1 or 6, further comprising at least one component selected from the group consisting of a PCR additive, an enzyme, and a replication accessory factor.

17. The composition of claim 1, further comprising about 0.1-10% dimethyl sulfoxide.

18. The composition of any of claims 1, 2, or 3, wherein the proofreading DNA polymerase is Pfu and the non-proofreading DNA polymerase is Taq.

19. The composition of claim 3, wherein the proofreading DNA polymerase is Pfu and the non-proofreading DNA polymerase is Taq and the Pfu:Taq ratio is about 1:1, as determined by units of polymerase activity.

20. The composition of claim 2, wherein the proofreading DNA polymerase is Pfu and the non-proofreading DNA polymerase is Taq and the Pfu:Taq ratio is greater than about 1:1, as determined by units of polymerase activity.

21. The composition of claim 20, wherein the Pfu:Taq ratio is about 2 to 3:1, as determined by units of polymerase activity.

22. The composition of claim 20, wherein the Pfu:Taq ratio is greater than about 3:1.

23. The composition of claim 1, wherein the proofreading DNA polymerase is a member of the archaeal DNA polymerase II family.

24. The composition of claim 23, wherein the archaeal DNA polymerase is P. furiosus pol II.

25. The composition of claim 4, wherein the buffer comprises about 20 to 70 mM Tricine with a pH of about 8.0 to 9.5 or about 10 to 70 mM Tris with a pH of about 8.0 to 9.5; 0 to less than about 16 mM (NH₄)₂SO₄; about 0.01 to 0.2% Tween-20 or Triton X-100; about 1.5 to 3 mM MgCl₂, MgSO₄, or C₄H₆O₄Mg; 0 to about 100 μg/ml bovine serum albumin; and 0 to about 4 mM dithiothreitol.

26. The composition of claim 4, wherein the buffer comprises about 20 to 70 mM Tricine with a pH of 8.4 to 9.2 or about 10 to 70 mM Tris with a pH of 8.4 to 9.2.

27. The composition of claim 4, wherein the buffer comprises 50 mM Tricine with a pH of 9.1; 8 mM (NH₄)₂SO₄; 0.1% Tween-20; 2.3 mM MgCl₂; 75 μg/ml nuclease-free bovine serum albumin; and 2 mM dithiothreitol.

28. The composition of claim 1, wherein the thermostable non-proofreading DNA polymerase is selected from the group consisting of Taq polymerase, Thermus flavus DNA polymerase I, Thermus thermophilus HB-8 DNA polymerase I, Thermophilus ruber DNA polymerase I, Thermophilus brokianus DNA polymerase I, Thermophilus caldophilus GK14 DNA polymerase I, Thermophilus filoformis DNA polymerase I, Bacillus stearothermophilus DNA polymerase I, Bacillus caldotonex YT-G DNA polymerase I, Bacillus caldovelox YT-F DNA polymerase I, and any other eubacterial DNA polymerase.

29. The composition of claim 1, wherein the thermostable proofreading DNA polymerase is selected from the group consisting of Pfu polymerase, Pwo polymerase from Pyrococcus woeseii, Deep Vent polymerase from Pyrococcus sp. GB-D, KOD polymerase from Thermococcus sp. KOD, Taq from Thermus aquaticus, Vent polymerase from Thermococcus litoralis, JDF-3 polymerase from Thermococcus sp. JDF-3, Pyrococcus abysii DNA polymerase, Pyrococcus horikoshii DNA polymerase, Pyrodictium occultum DNA polymerase, Archaeoglobus fulgidus DNA polymerase, Sulfolobus solfatanicus DNA polymerase, Sulfolobus acidocaldarium DNA polymerase, Thermococcus species 9 degrees North-7 DNA polymerase, Thermococcus gorgonarius DNA polymerase, Methanobacterium thermoautotrophicum DNA polymerase, Methanococcus jannaschii DNA polymerase, Thermoplasma acidophilum DNA polymerase, and any other archaeal DNA polymerase.

30. A method for amplifying a nucleic acid comprising employing the composition of any of claims 1-6 in an amplification reaction.

31. The method of claim 30, wherein the amplification reaction is polymerase chain reaction.

32. The method of claim 30, wherein the amplification reaction comprises isothermal rolling circle amplification.

33. The method of claim 30, wherein the amplification reaction comprises strand displacement amplification.

34. A method for synthesizing a nucleic acid comprising employing the composition of any of claims 1-6 in a nucleic acid synthesis reaction.

35. A method of mutagenizing a nucleic acid comprising employing the composition of any of claims 1-6 when mutagenizing the nucleic acid.

36. A kit for amplifying, synthesizing, or mutagenizing nucleic acids comprising: (a) a thermostable non-proofreading DNA polymerase, (b) a thermostable proofreading DNA polymerase, and (c) a factor that substantially inhibits the incorporation of undesired nucleotides or analogs thereof into a DNA polymer.

37. The kit of claim 36, wherein the amount of the proofreading DNA polymerase is greater than the amount of non-proofreading DNA polymerase, as determined by units of polymerase activity.

38. The kit of claim 36, wherein the amount of the proofreading DNA polymerase is less than or about equal to the amount of non-proofreading DNA polymerase, as determined by units of polymerase activity.

39. The kit of claim 36, further comprising a buffer that enhances a polymerization reaction involving the DNA polymerases.

40. The kit of claim 36, wherein the amount of the proofreading DNA polymerase is greater than the amount of non-proofreading DNA polymerase, as determined by units of polymerase activity, and further comprising a buffer that enhances a polymerization reaction involving the DNA polymerases.

41. The kit of claim 36, wherein (a), (b), and (c) are separate prior to use in amplifying, synthesizing, or mutagenizing nucleic acids.

42. The kit of claim 36, wherein at least two of (a), (b), and (c) are combined.

43. The composition of claim 27, wherein the proofreading polymerase is Pfu and the non-proofreading polymerase is Taq in a Pfu:Taq ratio of about 2:1, and the factor is PEF.

44. The composition of claim 43, further comrprising about 3% to about 7% DMSO.

45. The composition of claim 16, wherein the replication accessory factor is selected from the group consisting of PCNA, RFC-P38, RFC-P55, RFA, and an archaeal helicase.

46. The composition of claim 1, further comprising more than one proofreading DNA polymerase.

47. The composition of claim 1, further comprising more than one non-proofreading DNA polymerase.

48. The composition of claim 1, further comprising more than one factor.


SEQUENCE LISTING (30 sequences)

SEQ ID NO: 1 - DNA, 30 bp, Artificial Sequence
  Description of Artificial Sequence PCR Primer
atgctattaaaaacatggacaacctcaagc

SEQ ID NO: 2 - DNA, 24 bp, Artificial Sequence
  Description of Artificial Sequence PCR Primer
cacaagggctactggttgccgatt

SEQ ID NO: 3 - DNA, 30 bp, Artificial Sequence
  Description of Artificial Sequence Primer
ctcagatatggccaaagatctatacacacc

SEQ ID NO: 4 - DNA, 30 bp, Artificial Sequence
  Description of Artificial Sequence PCR Primer
agcttcccaacgtgatcgcctttctcccat

SEQ ID NO: 5 - DNA, 30 bp, Artificial Sequence
  Description of Artificial Sequence PCR Primer
cagggcattgacagcagtcttctcctcagg

SEQ ID NO: 6 - DNA, 30 bp, Artificial Sequence
  Description of Artificial Sequence PCR Primer
tatggttatcaggaaacagtccaggatctc

SEQ ID NO: 7 - DNA, 30 bp, Artificial Sequence
  Description of Artificial Sequence PCR Primer
acagcaagaaagcgagcttagtgatacttg

SEQ ID NO: 8 - DNA, 34 bp, Artificial Sequence
  Description of Artificial Sequence PCR Primer
ccttcactgtctgcctaactccttcgtgtgttcc

SEQ ID NO: 9 - DNA, 34 bp, Artificial Sequence
  Description of Artificial Sequence PCR Primer
gcaggggtgctgcagaactctgagctgtacttcc

SEQ ID NO: 10 - DNA, 31 bp, Artificial Sequence
  Description of Artificial Sequence PCR Primer
tgtctccagcacacagcatgttgtcggtgac

SEQ ID NO: 11 - DNA, 22 bp, Artificial Sequence
  Description of Artificial Sequence PCR Primer
cagctggctgacattttcggtg

SEQ ID NO: 12 - DNA, 22 bp, Artificial Sequence
  Description of Artificial Sequence PCR Primer
ccgcctttacaatgtccccgac

SEQ ID NO: 13 - DNA, 21 bp, Artificial Sequence
  Description of Artificial Sequence PCR Primer
cctgaattttcggtgatgcct

SEQ ID NO: 14 - DNA, 23 bp, Artificial Sequence
  Description of Artificial Sequence PCR Primer
cctgttatcaagcactgcactgg

SEQ ID NO: 15 - DNA, 22 bp, Artificial Sequence
  Description of Artificial Sequence PCR Primer
gaatcagcgcacatggtacagc

SEQ ID NO: 16 - DNA, 20 bp, Artificial Sequence
  Description of Artificial Sequence PCR Primer
gcatcagtaagcgcattggc

SEQ ID NO: 17 - DNA, 21 bp, Artificial Sequence
  Description of Artificial Sequence PCR Primer
gtttgggttgtgctgttgctg

SEQ ID NO: 18 - DNA, 22 bp, Artificial Sequence
  Description of Artificial Sequence PCR Primer
cctgagggctcccagagagtgg

SEQ ID NO: 19 - DNA, 22 bp, Artificial Sequence
  Description of Artificial Sequence PCR Primer
ggtttagcacgaccacaacagc

SEQ ID NO: 20 - DNA, 23 bp, Artificial Sequence
  Description of Artificial Sequence PCR Primer
gaggagagcaggaaaggtggaac

SEQ ID NO: 21 - DNA, 23 bp, Artificial Sequence
  Description of Artificial Sequence PCR Primer
gaggtacagggttgaggctagtg

SEQ ID NO: 22 - DNA, 22 bp, Artificial Sequence
  Description of Artificial Sequence PCR Primer
gaaaataggagctcagctgcag

SEQ ID NO: 23 - DNA, 22 bp, Artificial Sequence
  Description of Artificial Sequence PCR Primer
ttggacagggatgaggaataac

SEQ ID NO: 24 - DNA, 32 bp, Artificial Sequence
  Description of Artificial Sequence PCR Primer
gagcaatggtcaaagtcaacgtcatccacagc

SEQ ID NO: 25 - DNA, 33 bp, Artificial Sequence
  Description of Artificial Sequence PCR Primer
catagcgaattcgcaaaacctttcgcggtatgg

SEQ ID NO: 26 - DNA, 33 bp, Artificial Sequence
  Description of Artificial Sequence PCR Primer
actacggaattccacggaaaatgccgctcatcc

SEQ ID NO: 27 - DNA, 18 bp, Artificial Sequence
  Description of Artificial Sequence PCR Primer
gacagtcactccggcccg

SEQ ID NO: 28 - DNA, 18 bp, Artificial Sequence
  Description of Artificial Sequence PCR Primer
cgacgactcgtggagccc

SEQ ID NO: 29 - DNA, 18 bp, Artificial Sequence
  Description of Artificial Sequence PCR Primer
gacagtcactccggcccg

SEQ ID NO: 30 - DNA, 18 bp, Artificial Sequence
  Description of Artificial Sequence PCR Primer
cgacgactcgtggagccc